(19) United States
(12) Patent Application Publication
(10) Pub. No.: US 2008/0167534 A1
(43) Pub. Date: Jul. 10, 2008

(54) Information collecting apparatus for collecting physiological parameters and method thereof

(75) Inventors: Shuenn-Tsong Young, Taipei City (TW); Chung-Wang Lee, Hsinchu (TW); Tsair Kao, Taipei City (TW); Chang-Yu Liu, Yuanlin Township (TW); Chih-Wei Hsu, Rende Township (TW); Xin-Hua Zhang, Sinhua Township (TW)

Correspondence Address: BACON & THOMAS, PLLC, 625 SLATERS LANE, FOURTH FLOOR, ALEXANDRIA, VA, 22314, US

(73) Assignee: National Yang-Ming University, Taipei City (TW)

(21) Appl. No.: 11/649,304

(22) Filed: Jan. 4, 2007

(51) Int. Cl. A61B 5/00

(52) U.S. Cl. 600/301

(57) ABSTRACT

The present invention discloses an information collecting apparatus for collecting physiological parameters and the method is applied in the information collecting apparatus. When the information collecting apparatus receives a user's physiological parameter and determines that the physiological parameter does fall within the range of a monitoring limit value registered in the information collecting apparatus, then the information collecting apparatus will send a notice to a management server, so that the personnel, organization or department at the management server is able to monitor the user's health condition and select the most appropriate time to take actions and provide appropriate medical treatments for the user.

### FIELD OF THE INVENTION

The present invention relates to an information collecting apparatus for collecting physiological parameters and its method, and more particularly to an information collecting apparatus for collecting physiological parameters and its method capable of monitoring the user's health condition, and selecting the most appropriate time to take actions and provide appropriate medical treatments for the user.

### BACKGROUND OF THE INVENTION

As medical science is well developed, medical services and hygiene are enhanced, and the world's average fertility rate is dropped gradually, our health condition and average life expectancy are improved significantly, and thus our population tends to be an ageing population. In the ageing population, related issues of the medical services and social welfares for ageing and chronic diseases arise. To cope with this trend and satisfy the patient requirement for healthcare service at home, the market demand of healthcare and remote homecare for elderly people and patients becomes increasingly higher.

In recent years, information technology keeps advancing, the applications of remote homecare and medical service become more diversified, and many developed countries aggressively conduct researches and promotions on remote homecare and medical service to solve the problems caused by the elderly population. These activities not only help medical institutes including hospitals or clinics to achieve the purposes of sharing resources and saving costs, but also effectively promote homecare and medical services to remote districts with insufficient medical resources.

In general, the so-called “remote homecare” or “remote medical service” connects a patient and a family, or a patient and a doctor at two different places and transmits information such as a patient's physiological parameters (including blood pressure, heartbeat or blood sugar level) to a family or a doctor through the Internet or telephone network technology, so as to compile and save the information as the patient's physiological parameter history data and determine the patient's condition to provide timely homecare or medical service when the patient is unwell or in a critical condition. Thus, remote homecare or remote medical services can overcome the limitations of time and space by shifting the medical treatment activities originally conducted in hospitals or clinics to the patient's home.

To create a good remote homecare or remote medical service system, it is desirable to create a remote homecare or remote medical service software that integrates texts, data, figures, images, sounds, audio/video contents, in addition to converting medial information in an electronic form and transmitting the information via a fast broadband telecommunication network. The invested research fund and manpower are huge, and thus the cost of using a remote homecare or remote medical service system remains very high and unaffordable by ordinary people. What is more, traditional remote homecare or remote medical service systems still have the following two drawbacks:

Firstly, the aforementioned systems are constructed between a patient and a doctor (or a patient and a family member) at two different fixed locations, and thus once if the patient leaves his/her location, the system will no longer be unable to monitor the patient's physiological conditions and will result in a large psychological burden to the patient. The patient dares not to leave the fixed location and is seemingly imprisoned at the place. As a result, the patient's health will be affected adversely, particularly for chronic disease patients.

Secondly, the patient's physiological parameters obtained by the monitoring system are sent and stored at the location of a specific doctor, hospital or clinic, such that if a patient falls ill in another district, the patient's personal history data of the related physiological parameters will not be available easily, and thus will delay the timing of the medical treatments or even may cause unredeemable regrets.

Therefore, it is an important subject for researchers and manufacturers to make improvements by finding a way of using an existing low-price broadband network and a low equipment cost to design an information collecting apparatus for collecting physiological parameters to achieve the purpose of remote homecare or remote medical services, such that a patient can have absolute decision making and control on the patient's physiological parameters and can record and save the history data of the physiological parameters into an external storage device.

### SUMMARY OF THE INVENTION

In view of the foregoing shortcomings of the prior art, the inventor of the present invention based on years of experience to conduct extensive researches and experiments, and finally developed and designed an information collecting apparatus for collecting physiological parameters and its method in accordance with the present invention, in hope of using this innovative idea to contribute to the general public.

Therefore, it is a primary objective of the present invention to provide a method for collecting physiological parameters, and the method is applied in an information collecting apparatus, such that when the information collecting apparatus receives a user's physiological parameter and determines that the physiological parameter does fall within a range of a monitoring limit value registered in the information collecting apparatus, then the information collecting apparatus will send a notice to a management server, so that the personnel, organization or department at the management server will assist monitoring the user's health condition, and select the most appropriate time to take actions and provide appropriate medical treatments to the user.

Another objective of the present invention is to provide an information collecting apparatus for collecting physiological parameter, which comprises a first signal transceiver unit, a monitoring unit, a processing unit and a second signal transceiver unit, wherein the first signal transceiver unit is used to receive a wireless signal and analyze a physiological parameter from the wireless signal, the monitoring unit comprises at least one monitoring limit value each corresponding to a physiological parameter, and the processing unit is connected to the first signal transceiver unit and the monitoring unit and searches for the monitoring limit value corresponding to the physiological parameter. When the processing unit determines that the physiological parameter does not fall within the range of the corresponding monitoring limit value, the second signal transceiver unit will send a notice to a remote management server, so that the personnel, organization or department of the management server is able to monitor the user's health condition and select the most appropriate time to take actions and provide appropriate medical treatments to the user.

To make it easier for our examiner to understand the objective, technical characteristics and effects of the present invention, preferred embodiments will be described with accompanying drawings as follows:

### BRIEF DESCRIPTION OF THE DRAWINGS

FIG. 1 is a schematic view of the structure of a remote homecare system in accordance with the present invention;

FIG. 2 is a schematic view of the structure of an information collecting apparatus in accordance with the present invention; and

FIG. 3 is a flow chart of an information collecting apparatus receiving a wireless signal that carries a physiological parameter, a source instrument number and a source identification code in accordance with the present invention.

### DETAILED DESCRIPTION OF THE PREFERRED EMBODIMENTS

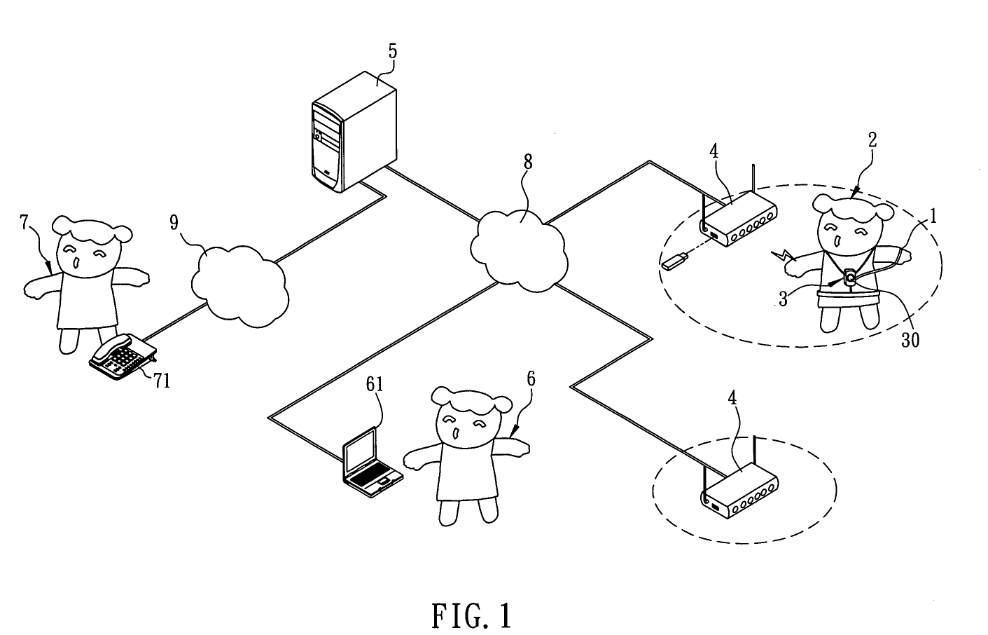

Referring to FIG. 1, a portable detecting instrument 1 (such as a sphygmomanometer or a blood sugar meter) is provided for measuring a physiological parameter (such as a blood pressure, a heartbeat or a blood sugar value) of a user 2 and storing an instrument number, and the portable detecting instrument 1 can be connected to a wireless transmitter module 3, and the wireless transmitter module 3 stores a source identification (ID) code (which is an exclusive personal identification code of the user 2) and receives a source instrument number of the portable detecting instrument 1, and a physiological parameter measured by the portable detecting instrument 1, and encapsulates the instrument number, physiological parameter and identification code into a wireless signal and sends out the wireless signal via a wireless transmission.

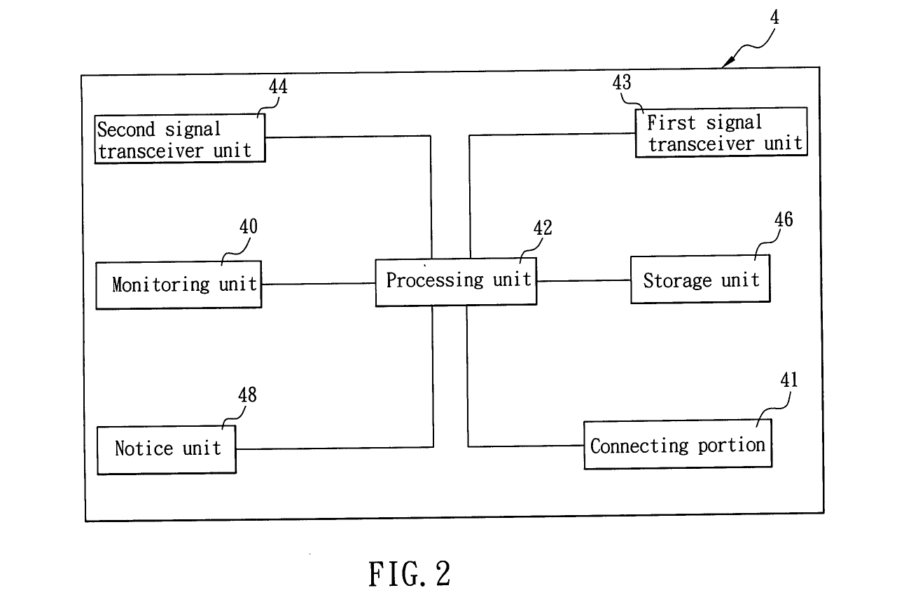

Referring to FIGS. 1 and 2, a method for collecting physiological parameters of the invention is applied in an information collecting apparatus 4, and the information collecting apparatus 4 stores a device identification code and comprises a monitoring unit 40, a processing unit 42, a first signal transceiver unit 43, a storage unit 46, a notice unit 48 and a second signal transceiver unit 44, wherein the storage unit 46 has at least one physiological information table for writing a physiological parameter therein, and each physiological information table is used to record various physiological parameters of a specific user 2, and the physiological parameters of each user 2 are sent from the same or different portable detecting instruments 1 to the information collecting apparatus 4 via the same or different wireless transmitter modules 3. In order to store various physiological parameters of different users 2 into different physiological information tables respectively, each physiological information table corresponds to a specific identification code and a specific instrument number, and the combination of an identification code and an instrument number corresponding to each physiological information table is the same as the combination of a source identification code and a source instrument number.

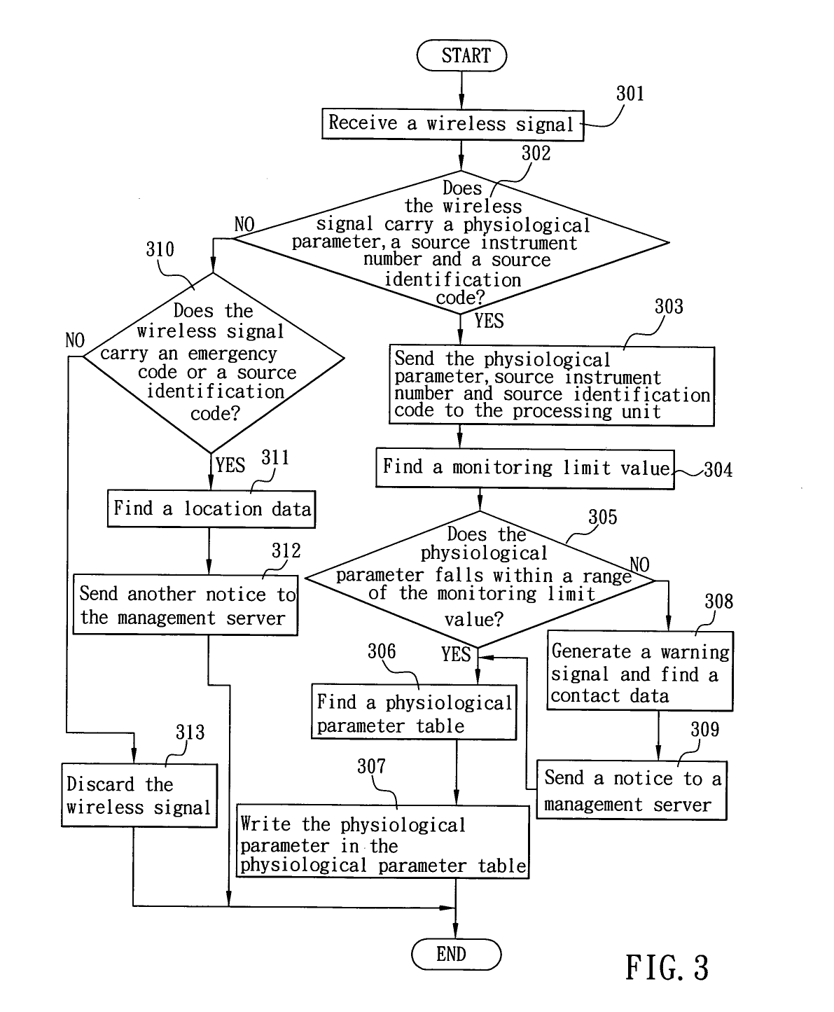

A plurality of monitoring limit values are pre-installed in the monitoring unit 40, and each monitoring limit value is set according to a physiological parameter of a user 2, and a physiological parameter of each user 2 is sent from the same or different portable detecting instruments 1 to the information collecting apparatus 4 via the same or different wireless transmitter modules 3 for monitoring various physiological parameters of a specific user 2, and each monitoring limit value corresponds to a specific identification code and a specific instrument number, and the combination of an identification code and an instrument number corresponding to the monitoring limit value is the same as the combination of a source identification code and a source instrument number. In addition, each monitoring limit value can be set by the user 2. For instance, a normal heartbeat is 60˜100 times per minute, and the monitoring limit value can be set to 60˜100 times per minute by the user, or the processing unit averages the physiological parameters of each physiological information table in a predetermined time period and multiplies the result by a risk parameter for computing the monitoring limit value and storing the monitoring limit value into the monitoring unit 40. Further, the notice unit 48 includes a location data (such as an IP address) of at least one management server 5, and each location data corresponds to a source identification code, and the information collecting apparatus 4 processes a procedure (as shown in FIG. 3) comprising the steps of:

Step (301): receiving a wireless signal;

Step (302): analyzing the wireless signal by a first signal transceiver unit 43 to determine whether or not the wireless signal carries a physiological parameter of a user 2, a source instrument number and a source identification code; if yes, then go to Step (303), or else go to Step (310);

Step (303): sending the physiological parameter, source instrument number and source identification code to the processing unit 42, if the wireless signal carries the physiological parameter, source instrument number and source identification code;

Step (304): finding a monitoring limit value in the monitoring unit 40 based on the source instrument number and source identification code by the processing unit 42, and the instrument number and identification code corresponding to the monitoring limit value are the same as the source instrument number and source identification code;

Step (305): determining whether or not a physiological parameter falls within a range of the monitoring limit value by the processing unit 42; if yes, then go to Step (306), or else go to Step (308);

Step (306): finding a physiological information table in the storage unit 46 by the processing unit 42, and the instrument number and identification code corresponding to the physiological information table are the same as the source instrument number and source identification code;

Step (307): writing the physiological parameter into the found physiological information table, and ending the procedure;

Step (308): indicating the physiological parameter as an abnormal physiological condition if the processing unit 42 determines that the physiological parameter does not fall within a range of the monitoring limit value, and then producing a warning signal, and finding a location data corresponding to the source identification code from the notice unit 48;

Step (309): producing a notice by the processing unit 42 based on the warning signal, source identification code and device identification code, and sending the notice to the second signal transceiver unit 44, and sending the notice by the second signal transceiver unit 44 to a management server 5 corresponding to the location data, and then going to Step (306);

The wireless transmitter module 3 further includes an emergency button 30 (as shown in FIG. 1) or builds in a sensor (not shown in the figure), wherein the emergency button 30 is provided for the user 2 to press when an emergency occurs, so that the wireless transmitter module 3 can produce an emergency code accordingly, and the sensor is provided for detecting an instant abnormal movement (such as falling down quickly or lying for a long time) of the user 2, so that the wireless transmitter module 3 can produce a wireless signal that carries an emergency code and a source identification code. Referring to FIG. 3, if the information collecting apparatus 4 determines that the wireless signal does not carry a physiological parameter of a user 2, a source instrument number and a source identification code, the information collecting apparatus 4 will process a procedure comprising the steps of:

Step (310): analyzing the wireless signal by the first signal transceiver unit 43 to determine whether or not the wireless signal carries an emergency code and a source identification code; if yes, then go to Step (311), or else go to Step (313);

Step (311): finding a location area corresponding to the source identification code from the notice unit 48 based on the source identification code by the processing unit 42 and finding a corresponding location data;

Step (312): producing another notice based on the emergency code, source identification code and device identification code by the processing unit 42 and sending the other notice to the second signal transceiver unit 44, and sending the other notice to a management server 5 corresponding to the location data by the second signal transceiver unit 44, and ending the procedure.

In the foregoing method, a user 2 can press the emergency button 30 when the user feels unwell, or the sensor detects an instant abnormal movement of the user 2, and then the information collecting apparatus 4 will issue another notice to the management server 5 to notice a computer 61 of a related organization 6 on the management database registered by the user 2 or a telephone (or mobile phone) 71 (as shown in FIG. 1) of a family member 7, so as to assist the related organization 6 or family member 7 to monitor the physiological condition of the user 2, and select the most appropriate time to take actions and provide medical treatments to the user 2.

Step (313): discarding the wireless signal if the wireless signal does not carry an emergency code and a source identification code, and then ending the procedure.

The information collecting apparatus 4 can receive different physiological parameters of each user 2 and write the physiological parameters into different physiological information tables. If the management server 5 (such as a management server) receives a notice from the information collecting apparatus 4, a precaution reporting mechanism will be started, and a contact data (such as an email address or a telephone number) of a related organization 6 (such as a hospital or a doctor) or a family member 7 of the user 2 corresponding to a source identification code will be registered by the user 2 in a management database of the management server 5, and the location (such as the user's home) of the corresponding device identification code registered in the management database will be sent by an email or a voice mail via the Internet 8 or a telephone network 9 (as shown in FIG. 1) to notice a computer 61 or a telephone 71 of a related organization 6 or family member 7 registered by the user 2. The email or voice mail includes the information of the location of the information collecting apparatus 4, so that the related organization 6 or family member 7 can locate the user 2 within the range of receiving the wireless signal by the information collecting apparatus 4.

In the present invention, the information collecting apparatus 4 further process the following procedure:

The processing unit 42 receives a plug-in signal, and the plug-in signal notices the information collecting apparatus 4 that an external storage device A (such as a flash disk) as shown in FIG. 1 is installed at a connecting portion 41 as shown in FIG. 2 for producing a storage signal based on the plug-in signal; and the processing unit 42 sends all present physiological information tables in the storage unit 46 to the external storage device A through the connecting portion 41 based on the storage signal and stores the physiological information tables in the external storage device A.

Referring to FIG. 2 for an information collecting apparatus for collecting physiological parameters of the present invention, the information collecting apparatus 4 stores a device identification code and includes a monitoring unit 40, a processing unit 42, a first signal transceiver unit 43, a notice unit 48 and a second signal transceiver unit 44. The first signal transceiver unit 43 receives a wireless signal and decodes the wireless signal to obtain a source identification code, a source instrument number and a physiological parameter carried by the wireless signal. The monitoring unit 40 includes a plurality of monitoring limit values corresponding to different identification codes and instrument numbers. The notice unit 48 includes a location data of at least one management server 5 (such as a management server). The processing unit 42 is connected to the first signal transceiver unit 43 and the monitoring unit 40 for receiving the source identification code and source instrument number, and finding a monitoring limit value in the monitoring unit 40 based on the source identification code and source instrument number, and the instrument number and identification code corresponding to the found monitoring limit value are the same as the source instrument number and source identification code. If the processing unit 42 determines that the received physiological parameter does not fall within the range of the found monitoring limit value, the physiological parameter will indicate an abnormal physiological condition, and a warning signal will be issued. Further, the processing unit 42 finds a location data corresponding to the source identification code in the notice unit 48 based on the source identification code, and produces a notice based on the warning signal, source identification code and device identification code, and sends the notice to the second signal transceiver unit 44. The second signal transceiver unit 44 sends the notice management server 5 to a corresponding to the location data, so that the personnel, organization or department of the management server 5 can assist monitoring the health condition of the user 2 and select the most appropriate time to take actions and provide appropriate medical treatments for the user 2.

In an information collecting apparatus of the invention, the first signal transceiver unit 43 will send the emergency code to the processing unit 42 if the first signal transceiver unit 43 decodes a wireless signal and the wireless signal carries an emergency code and source identification code, and then the processing unit 42 will find a location data corresponding to the source identification code based on the source identification code and produce another notice based on the emergency code, source identification code and device identification code and send the other notice to the second signal transceiver unit 44, and the second signal transceiver unit 44 will send the other notice to a management server 5 corresponding to the location data.

In an information collecting apparatus of the invention, the information collecting apparatus 4 further includes a storage unit 46 for storing at least one physiological information table, and each physiological information table has an instrument number and an identification code, and writes in a physiological parameter. If the processing unit 42 obtains the physiological parameter, the processing unit 42 will find a physiological information table having an instrument number and an identification code identical to the source instrument number and source identification code from the storage unit 46, and will write the physiological parameter into the found physiological information table.

In an information collecting apparatus of the invention, the information collecting apparatus 4 further includes a connecting portion 41 for installing an external storage device A (such as a flash disk), such that if the connecting portion 41 is installed to the external storage device A, the information collecting apparatus 4 will store all physiological information tables of the storage unit 46 into the external storage device A.

Since the present invention adopts an information collecting apparatus 4 to collect various physiological parameters of different users 2 and writes the physiological parameters into different physiological information tables, a user can carry the external storage device A containing the physiological information tables to a hospital or a clinic when the user 2 seeks for medical advices or treatments. After the hospital or clinic reads the physiological information tables, a diagnosis can be preformed for the user 2 immediately to avoid a delay of the timing for the medical treatments. If the physiological parameter does not fall within a range of the monitoring limit value, or the wireless signal carrying the emergency code is received, the information collecting apparatus 4 will send out the notice signal, so that the personnel, organization or department at the management server 5 can monitor the health condition of the user 2 and select the most appropriate time to take actions and provide appropriate medical treatments to the user 2. The user 2 also can personally set the monitoring limit value in the monitoring unit 40, so that the user 2 can have absolute decision making and control on the information collecting apparatus 4 to back up, edit and use the information as needed for emergency situations.

While the invention herein disclosed has been described by means of specific embodiments, numerous modifications and variations could be made thereto by those skilled in the art without departing from the scope and spirit of the invention set forth in the claims.

What is claimed is:

1. A method for collecting physiological parameters, applied to an information collecting apparatus, such that said information collecting apparatus processes a procedure comprising the steps of:
receiving a wireless signal;
analyzing said wireless signal to determine whether or not said wireless signal carries a user's physiological parameter, a source instrument number and a source identification code;
finding a predetermined monitoring limit value based on said source instrument number and said source identification code, when said wireless signal is determined to carry a physiological parameter, a source instrument number and a source identification code;
determining whether or not said physiological parameter falls within a range of said monitoring limit value;
producing a warning signal, and finding a location data corresponding to said source identification code, when said physiological parameter is determined to be not falling within the range of said monitoring limit value; and
producing a notice based on said warning signal, said source identification code and said device identification code, and sending said notice to a remote management server corresponding to said location data.

2. The method of clam 1, wherein before said information collecting apparatus determines that said wireless signal does not carries a user's physiological parameter, a source instrument number and a source identification code, said information collecting apparatus processes a procedure comprising the steps of:
analyzing said wireless signal to determine whether or not said wireless signal carries an emergency code and a source identification code;
finding a location data corresponding to said source identification code, when said wireless signal carries an emergency code and a source identification code; and
producing another notice based on said emergency code, said device identification code and said source identification code, and sending said other notice to a management server corresponding to said location data.

3. The method of claim 2, wherein said wireless signal will be discarded, when said information collecting apparatus determines that said wireless signal does not carry an emergency code and a source identification code.

4. The method of claim 1, wherein said physiological parameter will be written into said physiological information table and said source identification code will be found, when said information collecting apparatus determines that said physiological parameter falls within the range of said monitoring limit value, a physiological information table corresponding to said source instrument number.

5. The method of claim 1, wherein a physiological information table corresponding to said source instrument number and said source identification code is found, and said physiological parameter is written into said physiological information table, after said information collecting apparatus sends said notice to a remote management server.

6. An information collecting apparatus for collecting physiological parameters, wherein said information collecting apparatus stores a device identification code, comprising:
a first signal transceiver unit, for receiving a wireless signal, and analyzing said wireless signal to obtain a source identification code, a source instrument number and a physiological parameter carried by said wireless signal;
a monitoring unit, having a plurality of monitoring limit values corresponding to different identification codes and instrument numbers respectively;
a notice unit, including at least one location data of a management server, and said location data corresponding to a source identification code;
a processing unit, coupled to said first signal transceiver unit and said monitoring unit, for receiving said source identification code and said source instrument number, and finding a monitoring limit value corresponding to an instrument number and an identification code which are identical to said source instrument number and source identification code respectively, such that when said processing unit determines said received physiological parameter not falling within the range of said found monitoring limit value, said processing unit will generate a warning signal and find a location data corresponding to said source identification code from said notice unit based on said source identification code to produce a notice based on said warning signal, said source identification code and said device identification code; and
a second signal transceiver unit, for receiving said notice, and sending said notice to a management server corresponding to said location data.

7. The apparatus of claim 6, wherein said first signal transceiver unit decodes said wireless signal and obtains an emergency code and a source identification code, and said first signal transceiver unit sends said emergency code to said processing unit;
said processing unit finds a location data corresponding to said source identification code and produces another notice based on said emergency code, said source identification code and said device identification code; and
said second signal transceiver unit receives another notice and sends said other notice to a management server corresponding to said location data.

8. The apparatus of claim 7, wherein said information collecting apparatus further comprises a storage unit for storing at least one physiological information table, and each physiological information table corresponds to an instrument number and an identification code and writes in a physiological parameter; and
when said processing unit obtains said physiological parameter, said processing unit finds a physiological information table in said storage unit and writes said physiological parameter into said found physiological information table, and an instrument number and an identification code corresponding to said found physiological information table are the same as said source instrument number and said source identification code.

9. The apparatus of claim 8, wherein said information collecting apparatus further comprises a connecting portion for installing an external storage device.
